(19) United States
(12) Patent Application Publication
(10) Pub. No.: US 2008/0228207 A1
(43) Pub. Date: Sep. 18, 2008

(54) Semi-automatic power assist for endoscopic vein dissector

(75) Inventors: Randal James Kadykowski, South Lyon, MI (US); David B. Maurer, Ann Arbor, MI (US)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignee: Terumo Cardiovascular Systems Corporation

(21) Appl. No.: 11/724,460

(22) Filed: Mar. 15, 2007

(51) Int. Cl. A61B 17/94

(52) U.S. Cl. 606/159

(57) ABSTRACT

An endoscopic vein dissector for removing a vein from a living body comprises a dissector tip adapted to penetrate between the vein and surrounding tissue. A handle is provided for manipulation by a device operator to guide the tip through an incision in the living body to the vicinity of the vein. A longitudinal rod is mounted between the dissector tip and handle such that the tip is movable with respect to the handle between a retracted position and an extended position. A driver applies motive power to move the dissector tip from the retracted position to the extended position. A control element is adapted to be activated by the device operator to assist in the desired penetration between the vein and the surrounding tissue.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to endoscopic dissection of blood vessels within the limb of a patient, and, more specifically, to power-assisted, semi-automatic operation of a dissector tip so as to ease the separation of blood vessels, such as saphenous veins, from surrounding tissue so they may be removed for use as a coronary artery bypass graft.

In connection with coronary artery bypass grafting (CABG), a blood vessel or vessel section, such as an artery or vein, is “harvested” (i.e., removed) from its natural location in a patient's body to use it elsewhere in the body. In CABG surgery, the blood vessel is used to form a bypass between an arterial blood source and the coronary artery that is to be bypassed. Among the preferred sources for the vessel to be used as the bypass graft are the saphenous veins in the legs and the radial artery in the arms.

Endoscopic surgical procedures for harvesting a section of a vein (e.g., the saphenous vein) subcutaneously have been developed in order to avoid disadvantages and potential complications of harvesting through a continuous incision (e.g., along the leg) for the full length of the desired vein section in order to provide adequate exposure for visualizing the vein and for introducing surgical instruments to sever, cauterize and ligate the tissue and side branches of the vein. One such minimally-invasive technique employs a small incision for locating the desired vein and for introducing one or more endoscopic harvesting devices. Primary dissection occurs by introduction of a dissecting instrument through the incision to create a working space and separate the vein from the surrounding tissue. Then a cutting instrument is introduced into the working space to severe the blood vessel from the connective tissue surrounding the section to be harvested and any side branches of the blood vessel. The branches may be clipped and/or cauterized.

In one typical procedure, the endoscopic entry site is located near the midpoint of the vessel being harvested, with dissection and cutting of branches proceeding in both directions along the vessel from the entry site. In order to remove the desired section of the blood vessel, a second small incision, or stab wound, is made at one end thereof and the blood vessel section is ligated. A third small incision is made at the other end of the blood vessel section which is then ligated, thereby allowing the desired section to be completely removed through the first incision. Alternatively, only the first two incisions may be necessary if the length of the endoscopic device is sufficient to obtain the desired length of the blood vessel while working in only one direction along the vessel from the entry point.

An example of a commercially available product for performing the endoscopic vein harvesting described above is the VirtuoSaph™ Endoscopic Vein Harvesting System from Terumo Cardiovascular Systems Corporation of Ann Arbor, Mich. Endoscopic vein harvesting systems are shown in U.S. Pat. No. 6,660,016 to Lindsay and U.S. patent application publication 2005/0159764A1 in the name of Kasahara et al, both of which are incorporated herein by reference in their entirety.

The dissector tool typically comprises a longitudinal stainless steel rod with a tip at one end and an operator handle at the other. The tip is tapered to a blunt end and is made of transparent plastic. An optical cable inserted through the hollow handle and hollow rod abuts the tip to allow for endoscopic viewing during dissection. The dissection proceeds along the perimeter of the vein being harvested to separate it from the surrounding tissue and to expose the side branches of the vein so that they can be severed with a cutting tool.

During dissection, the operator grasps the handle and pushes the rod and tip into the limb. The force required to separate the tissue can become sufficiently large to cause significant effort and strain by the operator. The repetitive nature of the motions of the hand, elbow, and shoulder can lead to fatigue or pain for the operator. Thus, it would be desirable to make dissection less physically demanding on the operator.

Any inefficiency in the dissection process lengthens the amount of time required for the vein harvesting. A quicker, more efficient procedure would lead to improved patient outcomes and better use of surgical resources.

### SUMMARY OF THE INVENTION

The present invention overcomes the foregoing disadvantages with a semi-automatic dissector unit having a tip that can be driven (e.g., pneumatically) from a retracted position to an extended position by manually actuating a control element or. pushbutton. By first aligning the dissector parallel with the vein, the extension force performs the tissue separation with reduced effort by the operator. In addition to reducing operator strain and speeding up the procedure, the use of a semi-automatic power assist improves the overall precision of the tissue dissection.

In one aspect of the invention, an endoscopic vein dissector for removing a vein from a living body comprises a dissector tip adapted to penetrate between the vein and surrounding tissue. A handle is provided for manipulation by a device operator to guide the tip through an incision in the living body to the vicinity of the vein. A longitudinal rod is mounted between the dissector tip and handle such that the tip is movable with respect to the handle between a retracted position and an extended position. A driver applies motive power to move the dissector tip from the retracted position to the extended position. A control element is adapted to be activated by the device operator to assist in the desired penetration between the vein and the surrounding tissue.

### BRIEF DESCRIPTION OF THE DRAWINGS

FIG. 1 is an illustration of the removal of the saphenous vein from the leg of a patient.

FIG. 2 is a side view of a prior art dissector unit.

FIG. 3 is a side view of a prior art cutting tool.

FIG. 4 is a partial cross-sectional view of the dissection of a blood vessel using a prior art dissector.

FIG. 5 is a partial cross-sectional view of dissection using the semi-automatic dissector unit of the present invention.

FIG. 6 is a partial cross-sectional view of a first embodiment of the semi-automatic dissector.

FIG. 7 shows the dissector tip in its extended position.

FIG. 8 is a partial cross-sectional view of a second embodiment of the semi-automatic dissector.

FIG. 9 is a diagram of the dissector system including a fixture for bracing the dissector during power-driven extension of the dissector tip.

### DETAILED DESCRIPTION OF PREFERRED EMBODIMENTS

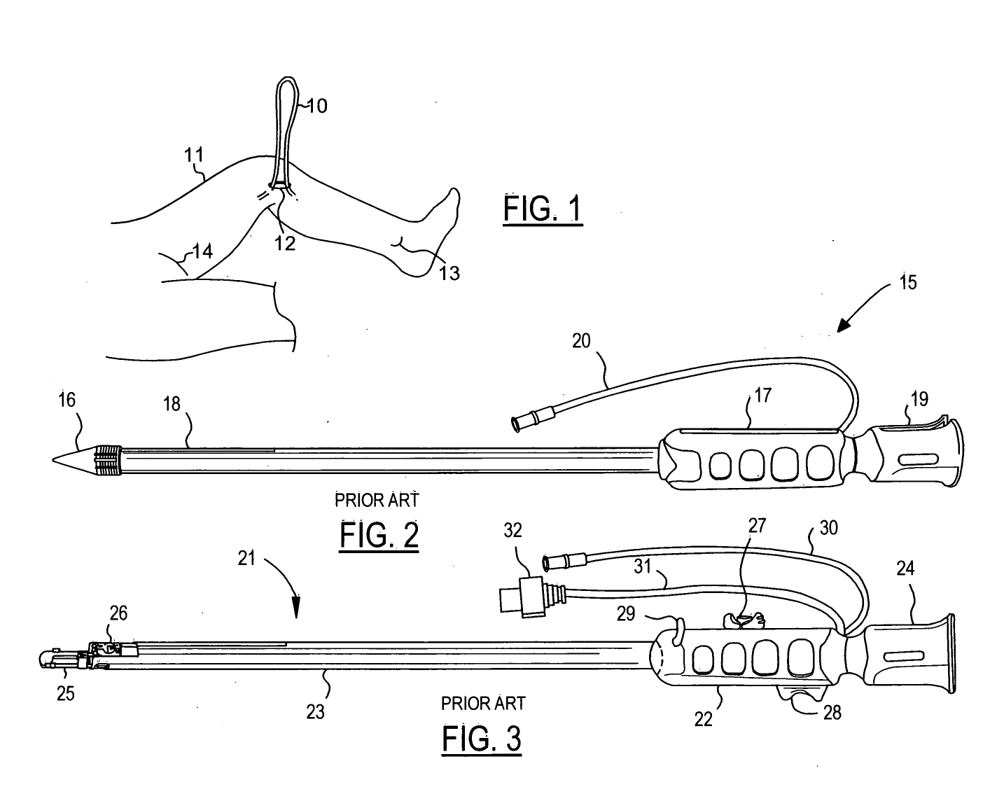

Referring now to FIG. 1, a saphenous vein 10 is being removed from a patient's leg 11 through an incision 12. During harvesting, main vessel 10 is severed from side branches extending from vessel 10 and then opposite ends of vessel 10 are cut at stab wounds 13 and 14 to free it for removal. The present invention may also be employed with blood vessels harvested using different surgical methods or from different areas of the patient's body.

Referring to FIG. 2, a disposable dissector unit 15 is shown of a known type for endoscopic dissection of a saphenous vein or other vessel by insertion through an initial incision and then pressing a dissector tip 16 into the fat along the direction of the vessel to separate it from adjacent tissue. Dissector unit 15 has a handle 17 connected to a longitudinal rod 18 having dissector tip 16 at its distal end. A receiver 19 at the end of handle 17 receives an endoscope and optical cable (not shown) for extending through rod 18 to dissector tip 16 which is transparent in order to allow visualization of the vessel and surrounding tissue. An insufflation tube 20 is connected to a source of CO₂ gas for filling the cavity adjacent the vessel as it is being formed in a conventional manner (i.e., the CO₂ gas passes through rod 18 to a release hole in or near tip 16).

After initial blunt dissection around the vein, a harvester rod 21 as shown in FIG. 3 is used to grasp the vessel being dissected and to sever any branches or connective tissue connecting to the vessel. Harvester rod 21 has a handle 22 connected to an elongated sleeve member 23 and an endoscope receiver 24. At the distal end of sleeve 23 are a V-keeper 25 for retaining the vessel being dissected and a V-cutter 26 for severing branches. V-keeper 25 is manipulated by V-keeper buttons 27 on handle 22. V-cutter 26 is extended or retracted by manipulating a V-cutter extender button 28 on handle 22. An endoscope wiper lever 29 is provided on handle 22 for controlling a wiper that clears the end of the endoscope when the endoscope optics become covered by material in the body cavity. An insufflator tube 30 can be connected to a source of gas such as CO₂ to deliver the gas to the distal end of sleeve 23. A bipolar cord 31 has a connector 32 at one end for connecting to a source of high frequency voltage, and includes conductors for supplying the voltage to electrodes on V-cutter 26 for cutting and cauterizing the branches and connective tissue.

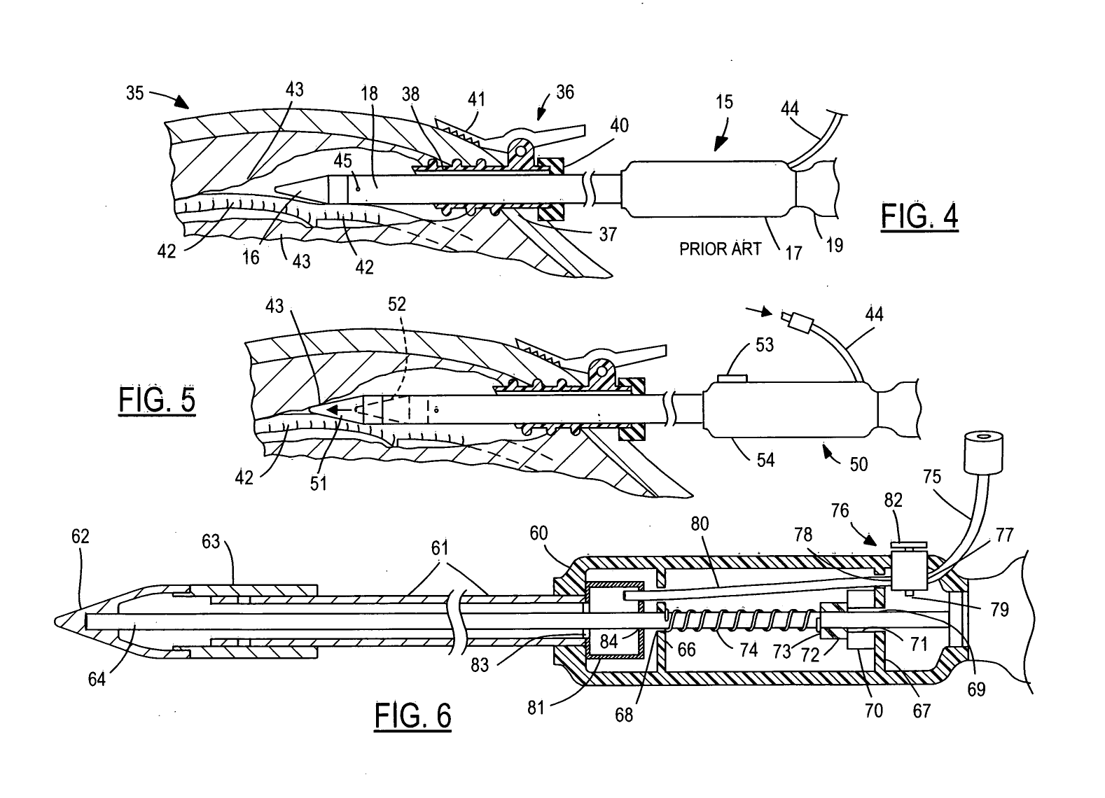

FIG. 4 is cross-sectional view showing a prior art dissector unit inserted subcutaneously under the lower limb 35 via a trocar 36 from a skin incision 37 in the direction of the inguinal region, for example. Trocar 36 comprises a cylindrical guide tube portion 38 for inserting rod 18, a sealing portion 40, and a fixing portion 41 for fixing the trocar to the skin. Tip 16 and rod 18 of dissector 15 are inserted subcutaneously under the skin via the guide tube portion 38 of trocar 36. An endoscope inserted through receiver 19 and handle 17 extends all the way to tip 16. Since the inserting direction of dissector 15 is along the direction of the blood vessel 42, the operator gradually inserts the dissector so as to dissect the peripheral tissue 43 from blood vessel 42 while viewing the endoscope image. By advancing dissector tip 16 along the inserting direction, the portion of blood vessel 11 leading to the inguinal region is gradually dissected and then a further portion of blood vessel 42 may be dissected in the opposite direction from incision 37 toward the ankle.

An insufflation gas (e.g., carbon dioxide) may be fed from an air feed tube 44 connected to handle 17. The gas is discharged from an opening 45 at a front end portion of rod 18. As blood vessel 42 is dissected from the peripheral tissue, the CO₂ gas inflates the area between the dissected tissue and the blood vessel. Therefore, the field of view of the endoscope is opened wide and visible recognition is improved.

An improved, semi-automatic dissector unit 50 is shown in FIG. 5 for more efficiently separating surrounding tissue 43 from blood vessel 42. Pressurized carbon dioxide from tube 44 or some other power-driven means are used to extend dissector tip 51 from its retracted position shown in dashed lines 52 to its extended position shown in solid lines. A control element or push button 53 is provided on handle 54 to control application of the motive power to dissector tip 51. Thus, the operator maintains handle 54 stationary and in parallel alignment with blood vessel 42 and then actuates control element 53 to cause dissector tip 51 to automatically extend along blood vessel 42 to separate tissue 43 from it. After the operator releases control element 53 and dissector tip 51 retracts, the dissector unit can be repositioned through the newly dissected tissue and then activated again to dissect even more tissue. Thus, the dissector unit 50 can be more quickly and easily advanced along blood vessel 42 to perform the dissection.

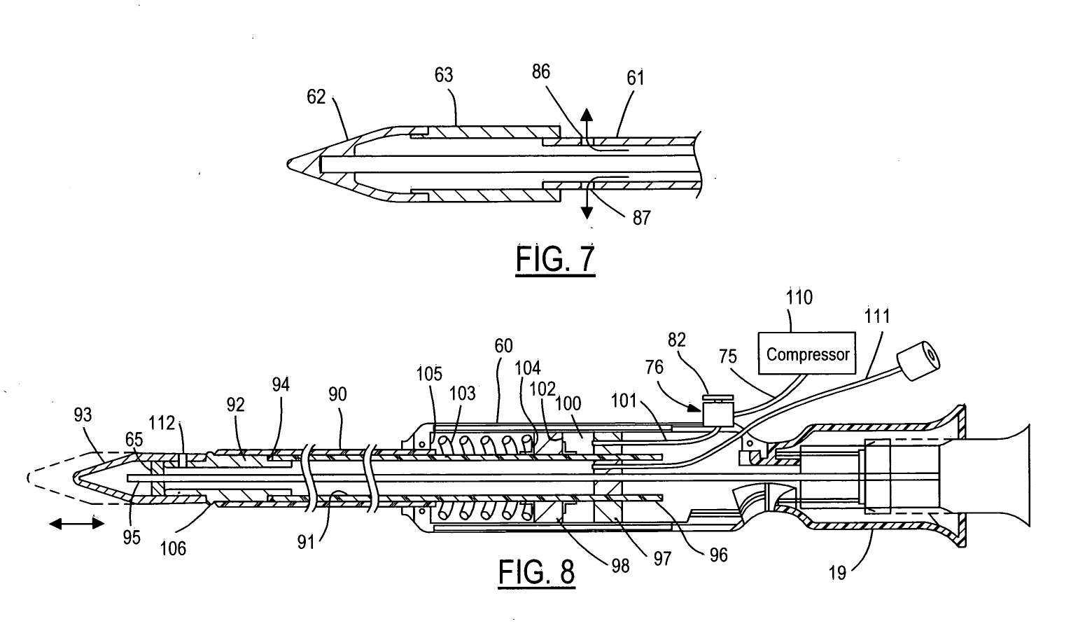

A first embodiment of the dissector unit is shown in FIG. 6. A handle 60 is rigidly mounted to a longitudinal hollow rod 61. A dissector tip 62 is mounted to a sleeve 63 which slides on rod 61. Tip 62 is transparent and is further mounted to a hollow central rod 64 which functions as an outer jacket for receiving an optical camera cable or an endoscope. Jacket 64 is slideably mounted within handle 60 so that tip 62 and sleeve 63 are slideable between a retracted position as shown in FIG. 6 and in extended position as shown in FIG. 7.

Handle 60 includes a first interior wall 66 and a second interior wall 67 having central apertures 68 and 69, respectively, for receiving slideable central rod 64. A spacer 70 is fixed to second wall 67 and has a central aperture 71 likewise receiving central rod 64. A flange 72 is fixed to central rod 64 and has a working surface 73 abutting one end of a return spring 74 concentrically mounted on central rod 64. The other end of return spring 74 abuts first wall 66 so that working surface 73 receives the spring force from spring 74 for urging central rod 64 into the retracted position. Thus, when tip 62 is moved to the extended position, central rod 64 and flange 72 move to the left in FIG. 6 thereby compressing spring 74 between surface 73 and wall 66. Without an extension force acting on the tip, the retraction force of return spring 74 keeps the dissector tip in the retracted position toward handle 60 with flange 72 bearing against spacer 70 (i.e., spacer 70 acts as a stop for the retracted position).

Extension force to extend the tip/sleeve/central rod combination away from handle 60 is provided as follows. A pressurized gas such as the insufflation carbon dioxide gas is provided via a tube 75 to a control valve 76 which is manually operable. Control valve 76 is preferably a three-port, two-way valve having an inlet port 77 receiving the pressurized gas. An outlet port 78 is coupled to a tube 80 for supplying the pressurized gas to the interior of a chamber 81. Exhaust port 79 is coupled to ambient (i.e., atmospheric) pressure either directly outside of the handle or through an opening in the handle (e.g., as provided by the receiver). Control valve 76 could alternatively be separate from handle 60, such as an in-line valve remotely positioned along tube 75.

A manual push button 82 is mounted to control valve 76 for selecting either an open or closed state of valve 76. In the open state, the inlet port is coupled to the outlet port for supplying pressurized gas to chamber 81. In the closed state, outlet port 78 is coupled to exhaust port 79 so that any pressurized gas that is present will be removed from chamber 81. Preferably, control valve 76 is normally closed so that an open state is obtained only during the manual activation of push button 82.

Chamber 81 is shaped as a hollow disk and has an opening 83 to the interior of longitudinal rod 61. An aperture 84 in chamber 81 acts as a bearing and receives central rod 64 and preferably includes a seal in order to maintain pressure in chamber 81. Tip 62 has an interior working surface 85 which is physically oriented such that an extension force applied against working surface 85 by the pressurized gas tends to extend tip 62 away from handle 60. In other words, when pressurized gas is introduced into chamber 81 and the interior of longitudinal rod 61, the gas pressure against working surface 85 becomes greater than the opposing forces on the exterior of tip 62 causing it to move to the extended position. The extension force also overcomes the retraction force from return spring 74 so that tip 62 and sleeve 63 moves toward the left as shown in FIG. 7.

In order to insufflate the dissected regions with the carbon dioxide, one or more holes 86 and 87 may be provided in longitudinal rod 61 to allow the carbon dioxide gas to escape when the tip is in the extended position. It is desirable to keep the size and number of holes 86 and 87 to a minimum in order to avoid excessive pressure loss and oscillation of tip 62. Alternatively, a pneumatically separate line can be provided through the dissector unit to provide insufflation independently.

A second embodiment of the dissector unit is shown in FIG. 8 wherein like components are designated by like reference numerals. Handle 60 is connected to a fixed outer sleeve 90 which surrounds a slideable longitudinal rod 91. An adapter 92 couples rod 91 to a transparent dissector tip 93. Alternatively, adapter 92 and tip 93 could be a single piece. A seal 94 may be captured between rod 91 and adapter 92 to provide a seal for the sliding engagement between rod 91 and sleeve 90. Central rod 95 is hollow and acts as a jacket to receive an optical camera cable or an endoscope. Central rod 95 remains fixed in this embodiment. Tip 93 together with adapter 92 and rod 91 slide longitudinally between the retracted position shown by solid lines and extended position as shown in dashed lines. A support ring 65 is mounted between rod 95 and tip 93 and is fixed with respect to one of them and slidable with respect to the other, so that the end of rod 95 is supported.

Longitudinal rod 91 has projections 96 extending through a sealing block 97 mounted in the interior of handle 60. A piston 98 is fixedly mounted to longitudinal rod 91 creating a piston chamber 100 between sealing block 97 and piston 98. A tube 101 couples pressurized gas (or other fluid) from control valve 76 through sealing block 97 to piston chamber 100. Piston 98 has a first working surface 102 facing piston chamber 100 so that when pressurized gas is introduced into chamber 100 through valve 76, an extension force is applied against first working surface 102 causing piston 98 and longitudinal rod 91 to move toward the left into the extended position. A return spring 103 abuts a second working surface 104 of piston 98 and is compressed between second working surface 104 and an end surface 105 within handle 60. When control valve 76 is deactivated, pressurized gas from piston chamber 100 flows out through tube 101 to the exhaust port of control valve 76 so that a retraction force from return spring 103 applied against second working surface 104 causes piston 98 and longitudinal rod 91 to move back to the right into the retracted position. Adapter 92 includes a raised area 106 for abutting outer sleeve 90 to provide a stop limit for the retraction of tip 93.

As shown in FIG. 8, the pressurized gas for providing semi-automatic extension and retraction of the dissector tip may be a separate gas or other fluid provided by a compressor 110 as an alternative to using the same pressurized gas used to perform the insufflation function. Thus, a tube 111 may be provided for connecting to a standard carbon dioxide source so that the insufflation gas may be provided through sealing block 97 to the interior of longitudinal rod 91 and out a hole 112, provided in dissector tip 93. Although pressurized gas is shown herein as a source of motive power, the invention can use other means of driving the dissector tip such as an electric motor or solenoid.

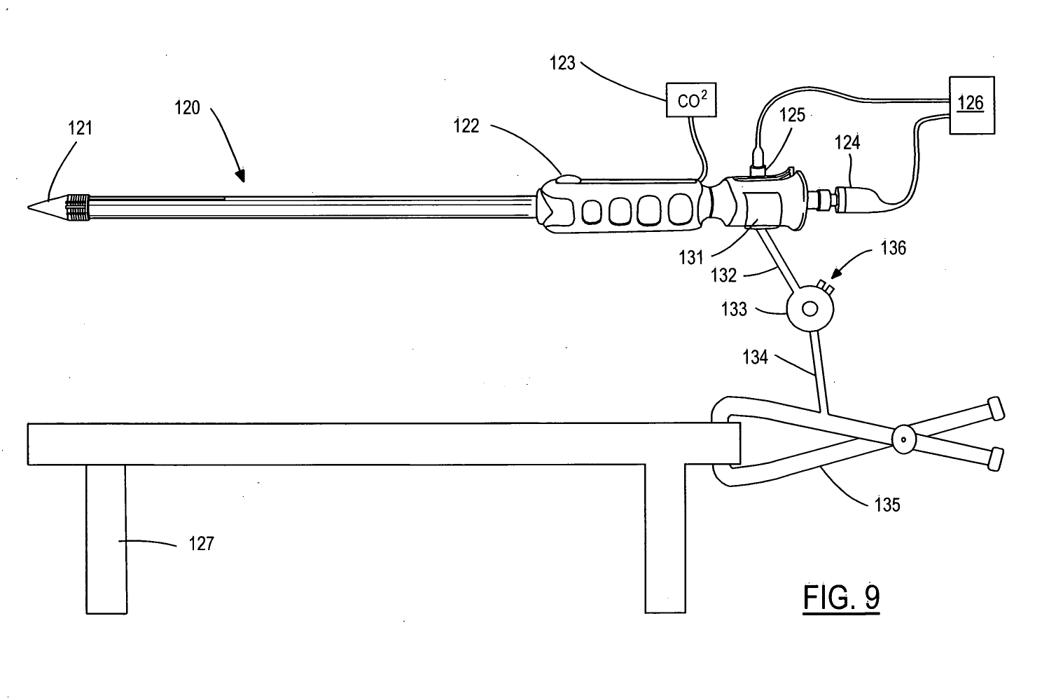

FIG. 9 shows a complete dissection system according to one embodiment of the invention. A dissector unit 120 with an extendable tip 121 under control of a push button 122 receives motive power for driving tip 121 from a supply of pressurized carbon dioxide 123. An endoscope 124 and a light source 125 are connected to the dissecting unit 120 and a controller 126. A patient having a vein being harvested is located on a table 127.

An operator utilizing the semi-automatic dissector unit may experience backlash from the extension forces when tip 121 is driven into the tissue of the patient. To address the backlash, a fixture 130 can be provided for bracing the operator handle against the extension force. Fixture 130 includes a mounting bracket 131 for attaching to either the handle or the receiver of dissector unit 120. An arm 132 couples mounting bracket 131 to a spindle 133 which is joined to an arm 134. A clip 135 is mounted to the end of arm 134 for mounting the fixture to a fixed object such as table 127. Spindle 133 has adjustable damping and allows manipulation of the position of dissector unit 120 relative to the vein being dissected under control of the operator. Adjustable damping of rotational movement of arms 132 and 134 around spindle 133 may be controlled by push buttons 136. For example, a first push button may lock spindle 133 into its current position and a second push button may unlock spindle 133 for free rotation. Alternatively, variable damping through hydraulic systems may be employed. Spindle 133 may include a ball and socket construction to allow multi-dimensional movement. Additional spindles connected in series along the arms can be used to add additional degrees of freedom for positioning dissector unit 120.

In operation, the damping response of the spindle(s) is switched to a low value to allow the operator to align the dissector tip and the longitudinal rod substantially parallel to a portion of the vein to be dissected. By selecting the low damping response, the location of the dissector unit is easily adjusted. The operator then selects a high damping response of the spindle 133 to substantially lock the dissector unit in position. Then the operator manually triggers extension of dissector tip 121 by pressing push button 122 on the handle. When the operator terminates the manual trigger by releasing push button 122, the dissector tip is retracted to the retracted position by the retraction force from the return spring. The operator then selects the low damping response again in order to manually re-align the dissector tip and longitudinal rod. Keeping the dissector tip and longitudinal rod substantially parallel to the next portion of the vein to be dissected, the operator then again selects the high damping response to lock the dissected unit in position. In this manner, the operator alternates manual triggering of the dissector tip extension with repositioning of the tip by appropriate selection of the damping response of the fixture to thereby dissect the surrounding tissue from the vein.

What is claimed is:

1. An endoscopic vein dissector for removing a vein from a living body, comprising:
a dissector tip adapted to penetrate between the vein and surrounding tissue;
a handle for manipulation by a device operator to guide the tip through an incision in the living body to the vicinity of the vein;
a longitudinal rod mounted between the dissector tip and handle such that the tip is movable with respect to the handle between a retracted position and an extended position;
a driver for applying motive power to move the dissector tip from the retracted position to the extended position; and
a control element adapted to be activated by the device operator to assist in the desired penetration between the vein and the surrounding tissue.

2. The endoscopic vein dissector of claim 1 further comprising:
a fixture mounted to the handle and adapted to be mounted to a fixed object for bracing the handle against reaction force during application of the motive power.

3. An endoscopic vein dissecting unit comprising:
an operator handle;
a longitudinal rod extending between a proximal end and a distal end, the proximal end being mounted to the operator handle;
a dissector tip mounted at the distal end of the longitudinal rod for longitudinal movement with respect to the operator handle, the dissector tip being adapted to penetrate between a vein and surrounding tissue in a living body from which the vein is to be harvested;
a first working surface arranged within the endoscopic vein dissecting unit such that an extension force applied against the first working surface tends to extend the dissector tip away from the operator handle;
a second working surface arranged within the endoscopic vein dissecting unit such that a retraction force applied against the second working surface tends to retract the dissector tip toward the operator handle;
a return spring acting against the second working surface to provide the retraction force; and
a manually-operated control valve for coupling a source of pressurized fluid against the first working surface to provide the extension force.

4. The unit of claim 3 further comprising a manual pushbutton;
wherein the control valve has an inlet port for coupling to the source of pressurized fluid, an outlet port for coupling to the first working surface, and an exhaust port for coupling to ambient pressure, and wherein the outlet port is selectably coupled to either the inlet port or the exhaust port in response to activation of the manual pushbutton.

5. The unit of claim 4 wherein the manual pushbutton is mounted to the operator handle.

6. The unit of claim 4 further comprising a fixture mounted to the operator handle and adapted to be mounted to a fixed object for bracing the operator handle against the extension force.

7. The unit of claim 6 wherein the fixture provides adjustable damping against movement of the handle.

8. The unit of claim 3 wherein the longitudinal rod is stationary with respect to the operator handle, and wherein the unit further comprises:
a central rod slidably received within the longitudinal rod, wherein the central rod has a distal end fixedly attached to the dissector tip and a collar for providing the second working surface, wherein the operator handle includes a first fixed bearing surface, wherein the return spring is compressed between the first fixed bearing surface and the second working surface on the collar, and wherein the operator handle includes a second fixed bearing surface for engaging the collar to provide a stop limit when the dissector tip is retracted by the retraction force.

9. The unit of claim 8 wherein said central rod comprises an optical cable jacket.

10. The unit of claim 8 further comprising a sleeve attached to the dissector tip slidably receiving the distal end of the longitudinal rod.

11. The unit of claim 10 wherein the longitudinal rod includes a gas passage which is covered by the sleeve when the dissector tip is retracted and which is uncovered by the sleeve when the dissector tip is extended, wherein the pressurized fluid flows through the gas passage when uncovered to insufflate tissue surrounding the dissector tip.

12. The unit of claim 3 further comprising:
a piston mounted to the longitudinal rod, wherein the piston has a first side for providing the first working surface; and
a sealed chamber receiving the piston.

13. A method of dissecting a vein from a living body using a dissector having a dissector tip connected to an operator handle via a longitudinal rod, the method comprising the steps of:
inserting the dissector tip through an incision in the living body to the vicinity of the vein;
aligning the dissector tip and longitudinal rod substantially parallel to a portion of the vein;
in response to manual triggering, extending the dissector tip with respect to the handle from a retracted position to an extended position with an extension force provided by a driver in order to separate surrounding tissue from the vein;
in response to termination of the manual trigger, retracting the dissector tip to the retracted position with a retraction force provided by a return spring; and
manually re-aligning the dissector tip and longitudinal rod substantially parallel to another portion of the vein and repeating the extension and retraction steps.

14. The method of claim 13 further comprising the step of:
bracing the operator handle in the substantially parallel position using a fixture that couples the operator handle to a fixed object.

15. The method of claim 14 wherein the fixture selectably provides a high damping response or a low damping response, the method further comprising the steps of:
selecting the low damping response when aligning the dissector tip and longitudinal rod substantially parallel to a portion of the vein;
selecting the high damping response prior to manually triggering the extending step; and
selecting the low damping response when manually re-aligning the dissector tip and longitudinal rod.
